(19) United States
(12) Patent Application Publication
(10) Pub. No.: US 2025/0140380 A1
(43) Pub. Date: May 1, 2025

(54) INFORMATION MANAGEMENT SERVER, INFORMATION SHARING SYSTEM AND STORAGE MEDIUM

(71) Applicant: Konica Minolta, Inc., Tokyo (JP)

(72) Inventor: Moeko HAGIWARA, Ichikawa-shi (JP)

(21) Appl. No.: 18/918,319

(22) Filed: Oct. 17, 2024

(30) Foreign Application Priority Data
Oct. 26, 2023 (JP) 2023-183921

(51) Int. Cl. G16H 30/20

(52) U.S. Cl. CPC G16H 30/20

(57) ABSTRACT

An information management server includes a hardware processor. The hardware processor obtains first medical information in a DICOM format of a first medical facility and second medical information in the DICOM format of a second medical facility, generates display information for a display of a user terminal to display the obtained first medical information and second medical information, and outputs the generated display information.

### CROSS-REFERENCE TO RELATED APPLICATIONS

The entire disclosure of Japanese Patent Application No. 2023-183921 filed on Oct. 26, 2023 is incorporated herein by reference in its entirety.

### BACKGROUND OF THE INVENTION

### Technical Field

The present invention relates to an information management server, an information sharing system and a storage medium.

### Description of Related Art

In the medical field, a method for storing a medical image in an electronic medical chart is not standardized. Therefore, it is difficult to manage medical images in an integrated manner in a personal health record (PHR) application.

There are many medical facilities that do not connect their electronic medical charts to a network environment outside their medical facilities, to which the electronic medical records belong, because it is a problem in terms of security to connect an electronic medical chart to a network environment outside its medical facility. Therefore, it is difficult to browse, outside a medical facility, medical information stored in an electronic medical chart in the medical facility.

With respect to the above-described problem, the following is described in Japanese Unexamined Patent Publication No. 2020-004354. Specifically, a patient takes a picture of his/her medical information, examples of which include an image, examination data and a summary displayed on a terminal of a doctor, with a mobile phone or the like that the patient uses. The patient can view his/her medical information at any time in the captured image.

### SUMMARY OF THE INVENTION

However, in the invention of Japanese Unexamined Patent Publication No. 2020-004354, it is cumbersome for the patient to take a picture of the medical information with a mobile phone or the like, and moreover, the quality of the taken picture not sufficient, so that the medical information may not be read accurately.

An object of the present invention is to provide an information management server, an information sharing system, and a storage medium that allow a patient to appropriately view medical information from a plurality of medical facilities.

To achieve at least one of the abovementioned objects, according to an aspect of the present invention, an information management server reflecting one aspect of the present invention includes a hardware processor that
obtains first medical information in a DICOM format of a first medical facility and second medical information in the DICOM format of a second medical facility,
generates display information for a display of a user terminal to display the obtained first medical information and second medical information, and
outputs the generated display information.

According to an aspect of the present invention, an information sharing system reflecting one aspect of the present invention includes:
the information management server; and
the user terminal including:
   the display; and
   a hardware processor that causes the display to display the first medical information and the second medical information satisfying a predetermined condition.

According to an aspect of the present invention, a non-transitory computer-readable storage medium reflecting one aspect of the present invention stores a program causing a computer of an information management server to perform:
obtaining first medical information in a DICOM format of a first medical facility and second medical information in the DICOM format of a second medical facility;
generating display information for a display of a user terminal to display the obtained first medical information and second medical information; and
outputting the generated display information.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram of a system configuration of an information sharing system of the present embodiment;

FIG. 2 is a block diagram illustrating a functional configuration of an information management server;

FIG. 3 is a block diagram illustrating a functional configuration of a user terminal;

FIG. 4 is a block diagram illustrating a functional configuration of a medical image management apparatus;

FIG. 5 is a block diagram illustrating a functional configuration of an information terminal;

FIG. 6 is a ladder chart showing an information sharing process;

FIG. 7 is a diagram illustrating an example of a medical information list screen;

FIG. 8 is a diagram showing an example of a medical facility selection screen; and

FIG. 9 is a diagram illustrating an example of a narrowing-down screen.

### DETAILED DESCRIPTION

In the following, an embodiment of an information sharing system according to the present invention will be described with reference to the drawings. However, the scope of the invention is not limited to the illustrated examples.

### [Configuration of Information Sharing System]

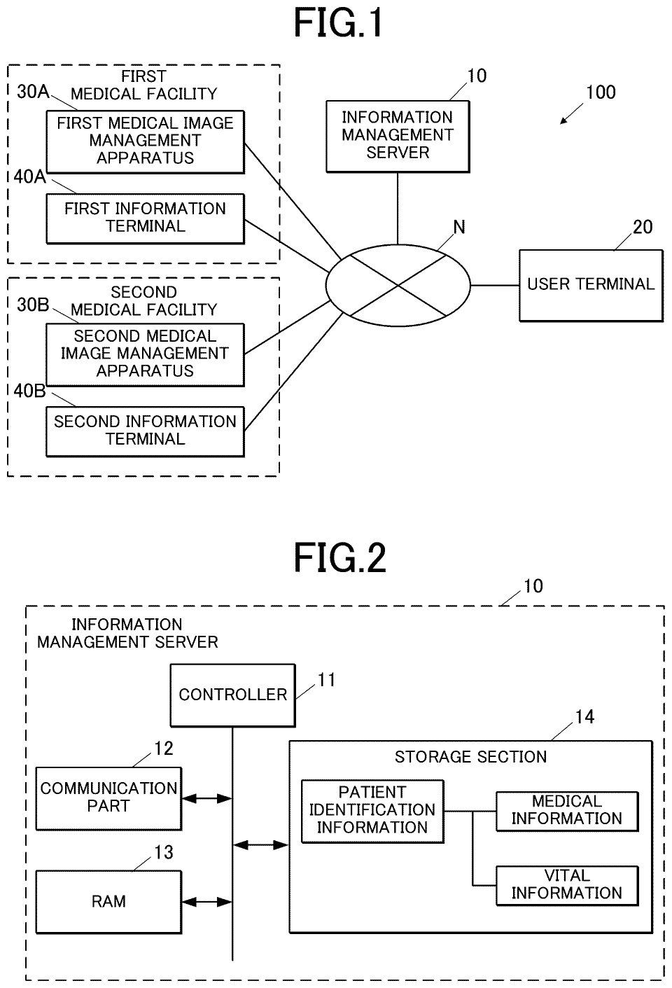

FIG. 1 illustrates a system configuration of an information sharing system 100.

As illustrated in FIG. 1, the information sharing system 100 includes an information management server 10, a user terminal 20, a first medical image management apparatus 30A, a second medical image management apparatus 30B, a first information terminal 40A, and a second information terminal 40B.

The information management server 10 is data-communicably connected to the user terminal 20, the first medical image management apparatus 30A, the second medical image management apparatus 30B, the first information terminal 40A, and the second information terminal 40B via a communication network N such as the Internet.

The information management server 10 is an apparatus that accumulates, manages and processes information on medical care of patients to be registered, from the user terminal 20, the first medical image management apparatus 30A, the second medical image management apparatus 30B, the first information terminal 40A, and the second information terminal 40B.

The information management server 10 is a cloud server provided in a cloud environment or a local server. For example, the information management server 10 is a PHR server.

The user terminal 20 is a terminal device such as a smartphone used by a user who is a patient.

The number of user terminals 20 is not limited to the example illustrated in FIG. 1.

The first medical image management apparatus 30A is an apparatus used by a medical professional (doctor, nurse, etc) belonging to a first medical facility.

The second medical image management apparatus 30B is an apparatus used by a medical professional (doctor, nurse, etc.) belonging to a second medical facility.

The first medical image management apparatus 30A and the second medical image management apparatus 30B are, for example, servers constituting a picture archiving and communication system (PACS).

Hereinafter, the first medical image management apparatus 30A and the second medical image management apparatus 30B may be collectively referred to as a medical image management apparatus(es) 30.

The first information terminal 40A is a device used by a medical professional (doctor, nurse, etc) belonging to the first medical facility.

The second information terminal 40B is a device used by a medical professional (doctor, nurse, etc) belonging to the second medical facility.

The first information terminal 40A and the second information terminal 40B are, for example, computer devices such as personal computers (PCs) or tablet terminals. Alternatively, the first information terminal 40A and the second information terminal 40B may be electronic medical chart apparatuses.

Hereinafter, the first information terminal 40A and the second information terminal 40B may be collectively referred to as an information terminal(s) 40.

### [Configuration of Information Management Server]

FIG. 2 illustrates a functional configuration of the information management server 10.

As illustrated in FIG. 2, the information management server 10 includes a controller 11 (hardware processor, a communication part 12, a RAM 13, a storage section 14, and the like, and these parts of the information management server 10 are connected by a bus.

The controller 11 includes a central processing unit (CPU) and the like, and comprehensively controls processing operation of each part of the information management server 10. To be specific, the CPU reads various process programs stored in the storage section 14, develop the programs in the RAM 13, and perform various processes in cooperation with the programs.

The communication part 12 includes a network interface or the like, and transmits and receives data to and from an external device connected via the communication network N.

The RAM 13 forms a work area for temporarily storing various types of information read from the storage section 14 in the various processes executed and controlled by the controller 11. The various types of information include various programs, input data, output data, parameters, and the like.

The storage section 14 includes a hard disk drive (HDD), a nonvolatile semiconductor memory or the like.

The storage section 14 stores various process programs, parameters and files necessary for executing the programs, and the like.

The storage section 14 stores, for each information for identifying a patient (patient identification information), medical information, vital information and the like on a patient.

The patient identification information is, for example, a patient ID.

The medical information is information input by a medical professional belonging to a medical facility via the medical image management apparatus 30 and/or the information terminal 40. Alternatively, the medical information is information automatically imported from a system in a medical facility to the medical image management apparatus 30 and/or the information terminal 40.

The medical information includes medical images held by the medical image management apparatus 30 and examination result information and diagnosis result information held by the information terminal 40.

A medical image is an image obtained by imaging a patient with radiation or the like in a medical facility. The medical image data of the present embodiment is data in the digital imaging and communications in medicine (DICOM) format. The medical image in the DICOM format includes an image portion and a supplementary information portion. Actual data as a medical image is written in the image portion, and various kinds of supplementary information regarding the medical image are written in the supplementary information portion.

The examination result information is information on an examination that a patient has undergone, and includes examination items, an examination result, and the like.

The diagnosis result information is information on a diagnosis that a patient has received, and includes diagnosis items, a diagnosis result, and the like.

The vital information is information that a patient inputs via the user terminal 20. Alternatively, the vital information is information automatically taken into the user terminal 20 from a vital measurement apparatus that measures vital information.

The vital information includes, for example, a blood glucose level, a blood pressure, a heart rate, a body temperature, and a weight.

### [Configuration of User Terminal]

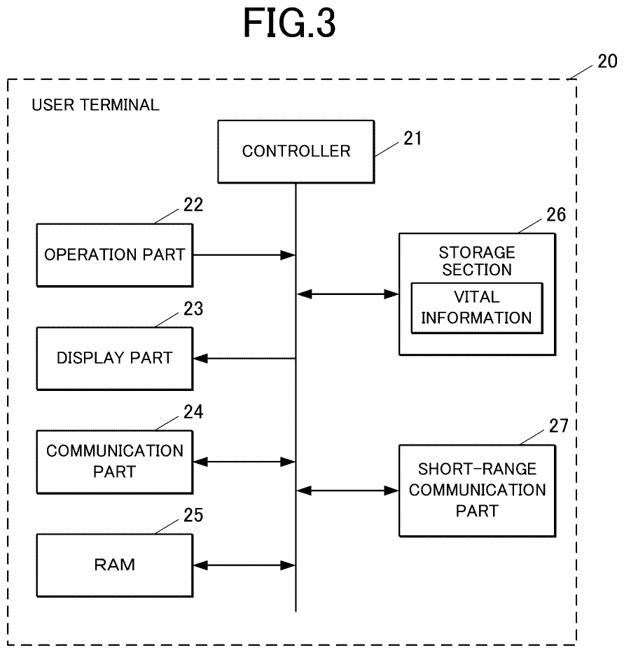

FIG. 3 illustrates a functional configuration of the user terminal 20.

As illustrated in FIG. 3, the user terminal 20 includes a controller 21 (hardware processor), an operation part 22, a display part 23 (display), a communication part 24, a RAM 25, a storage section 26, and a short-range communication part 27. These parts of the user terminal 20 are connected by a bus.

The controller 21 includes a CPU and the like, and comprehensively controls the processing operation of each part of the user terminal 20. To be specific, the CPU reads various process programs stored in the storage section 26 in response to operation signals input from the operation part 22 or instruction signals received by the communication part 24. The CPU develops the read various process programs into the RAM 25, and performs various processes in cooperation with the programs.

The operation part 22 includes operation keys such as a power key for turning on and off the power, a touch screen laid on the display part 23, and the like.

The operation part 22 outputs, to the controller 21, an operation signal corresponding to each operation key, and an operation signal corresponding to a position touched by an operator's finger or the like.

The display part 23 includes a liquid crystal display (LCD) or the like.

The display part 23 displays various screens in accordance with instructions of display signals input from the controller 21.

The communication part 24 includes a network interface or the like, and transmits and receives data to and from an external device connected via the communication network N.

The communication part 24 performs wireless communication using a mobile phone line or the like, and transmits and receives data to and from an external device connected via the communication network N.

The RAM 25 forms a work area for temporarily storing various types of information read from the storage section 26 in the various processes executed and controlled by the controller 21. The various types of information include various programs, input data, output data, parameters, and the like.

The storage section 26 includes an HDD, a nonvolatile semiconductor memory or the like, and stores various process programs and parameters and files necessary for executing the programs.

The storage section 26 stores vital information.

The vital information is input by a patient via the operation part 22 or obtained from the vital measurement apparatus via the short-range communication part 27.

The short-range communication part 27 performs data communication with an external device via a transmission/reception antenna for near field communication (NFC), Bluetooth® communication, or the like. The short-range communication part 27 receives, for example, vital information from the vital measurement apparatus.

### [Configuration of Medical Image Management Apparatus]

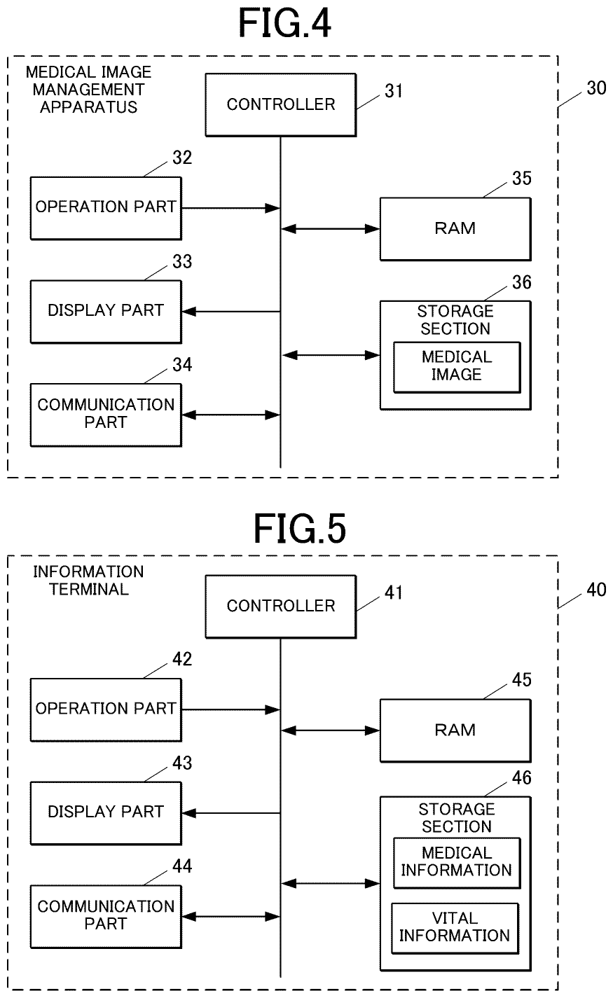

FIG. 4 shows a functional configuration of the medical image management apparatus 30.

As illustrated in FIG. 4, the medical image management apparatus 30 includes a controller 31, an operation part 32, a display part 33, a communication part 34, a RAM 35, and a storage section 36. These parts of the medical image management apparatus 30 are connected by a bus.

The controller 31, the display part 33, and the RAM 35 of the medical image management apparatus 30 are the same as the controller 21, the display part 23, and the RAM 25 of the user terminal 20, respectively, and therefore description thereof is omitted.

The operation part 32 includes a keyboard having cursor keys, character input keys, various function keys, and the like, and a pointing device such as a mouse.

The operation part 32 outputs, to the controller 31, an operation signal input by a key operation on the keyboard or a mouse operation.

The communication part 34 includes a network interface or the like, and transmits and receives data to and from an external device connected via the communication network N.

The storage section 36 includes an HDD, a nonvolatile semiconductor memory or the like, and stores various process programs and parameters and files necessary for executing the programs.

The storage section 36 stores medical images that are medical information.

The medical images are automatically obtained from, for example, an imaging apparatus (not illustrated) in a medical facility.

The storage section 36 of the first medical image management apparatus 30A stores a first medical image which is first medical information. The storage section 36 of the second medical image management apparatus 30B stores a second medical image which is second medical information.

### [Configuration of Information Terminal]

FIG. 5 shows a functional configuration of the information terminal 40.

As illustrated in FIG. 5, the information terminal 40 includes a controller 41, an operation part 42, a display part 43, a communication part 44, a RAM 45, and a storage section 46. These parts of the information terminal 40 are connected by a bus.

The controller 41, the display part 43, and the RAM 45 of the information terminal 40 are the same as the controller 21, the display part 23, and the RAM 25 of the user terminal 20, respectively, and therefore description thereof is omitted.

The operation part 42 includes a keyboard having cursor keys, character input keys, various function keys, and the like, and a pointing device such as a mouse.

The operation part 42 outputs, to the controller 41, an operation signal input by a key operation on the keyboard or a mouse operation.

In a case where the information terminal 40 is a tablet terminal or the like, the operation part 42 includes operation keys such as a power key for turning on/off the power, a touch screen laid on the display part 43, and the like. The operation part 42 in this case outputs, to the controller 41, an operation signal corresponding to each operation key, and an operation signal corresponding to a position touched by an operator's finger or the like.

The communication part 44 includes a network interface or the like, and transmits and receives data to and from an external device connected via the communication network N.

The storage section 46 includes an HDD, a nonvolatile semiconductor memory or the like, and stores various process programs and parameters and files necessary for executing the programs.

The storage section 46 stores examination result information, diagnosis result information and the like, which are medical information other than medical images.

The medical information other than the medical image may be input to the information terminal 40 by a medical professional, or may be automatically obtained from a system in a medical facility.

The storage section 46 of the first information terminal 40A stores first medical information other than the first medical image. The storage section 46 of the second information terminal 40B stores second medical information other than the second medical image.

The storage section 46 stores patient's vital information received from the information management server 10.

### [Operation of Information Sharing System]

Next, the operation in the information sharing system 100 will be described.

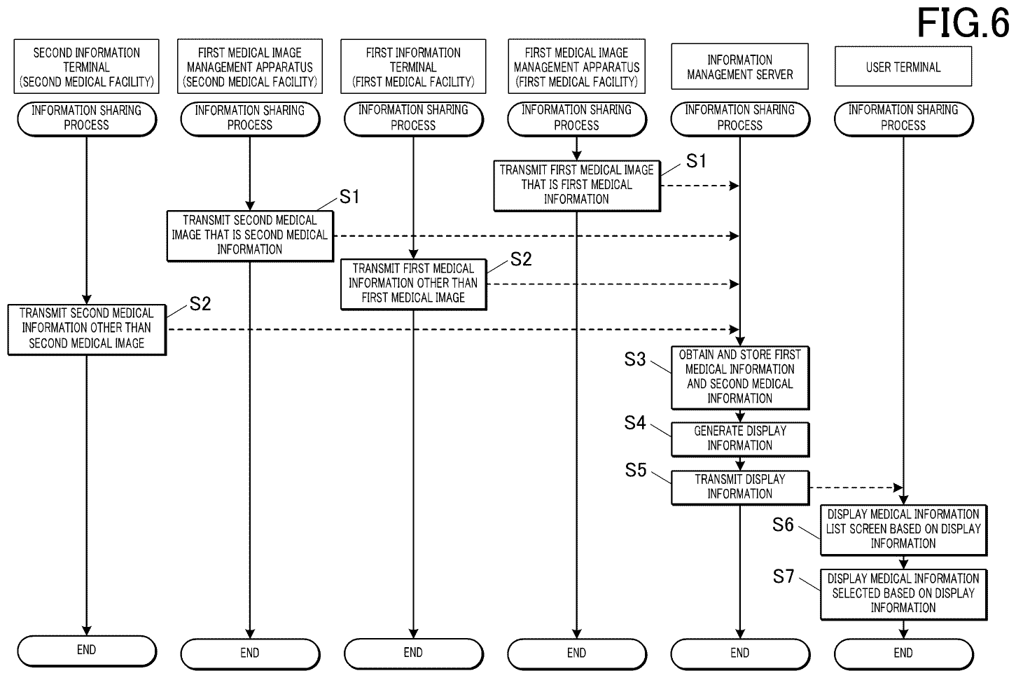

The apparatuses or the like included in the information sharing system 100 execute an information sharing process illustrated in FIG. 6.

### (Information Sharing Process)

The controller 31 of the first medical image management apparatus 30A transmits the first medical image that is the first medical information on the patient using the user terminal 20, to the information management server 10 (Step S1). The controller 31 of the second medical image management apparatus 30B transmits the second medical image that is the second medical information on the patient using the user terminal 20, to the information management server 10 (Step S1).

Next, the controller 41 of the first information terminal 40A transmits the first medical information other than the first medical image of the patient using the user terminal 20 to the information management server 10 (Step S2). The controller 41 of the second information terminal 40B transmits the second medical information other than the second medical image of the patient using the user terminal 20 to the information management server 10 (Step S2).

The controller 11 of the information management server 10 receives the first medical image transmitted from the first medical image management apparatus 30A and the second medical image transmitted from the second medical image management apparatus 30B, and stores them in the storage section 14. The controller 11 receives the first medical information other than the first medical image transmitted from the first information terminal 40A and the second medical information other than the second medical image transmitted from the second information terminal 40B, and stores them in the storage section 14. That is, the controller 11 obtains the first medical information and the second medical information (Step S3).

In other words, the controller 11 obtains the first medical information in the DICOM format from the first medical image management apparatus 30A of the first medical facility and obtains the second medical information in the DICOM format from the second medical image management apparatus 30B of the second medical facility. The controller 11 functions as an obtainment section.

Next, the controller 11 generates display information for the display part 23 of the user terminal 20 to display the first medical information and the second medical information obtained in Step S3 on t (Step S4). The controller 11 functions as a generation section. Specifically, the display information is information for the display part 23 to display a medical information list screen. The display information is information for the display part 23 to display a screen for displaying medical information selected by the user from the medical information list screen.

Next, the controller 11 outputs the display information generated in Step S4 by transmitting it to the user terminal 20 (Step S5). The controller 11 functions as an output section.

The controller 21 of the user terminal 20 receives the display information from the information management server 10, and causes the display part 23 to display the medical information list screen on the basis of the received display information (Step S6).

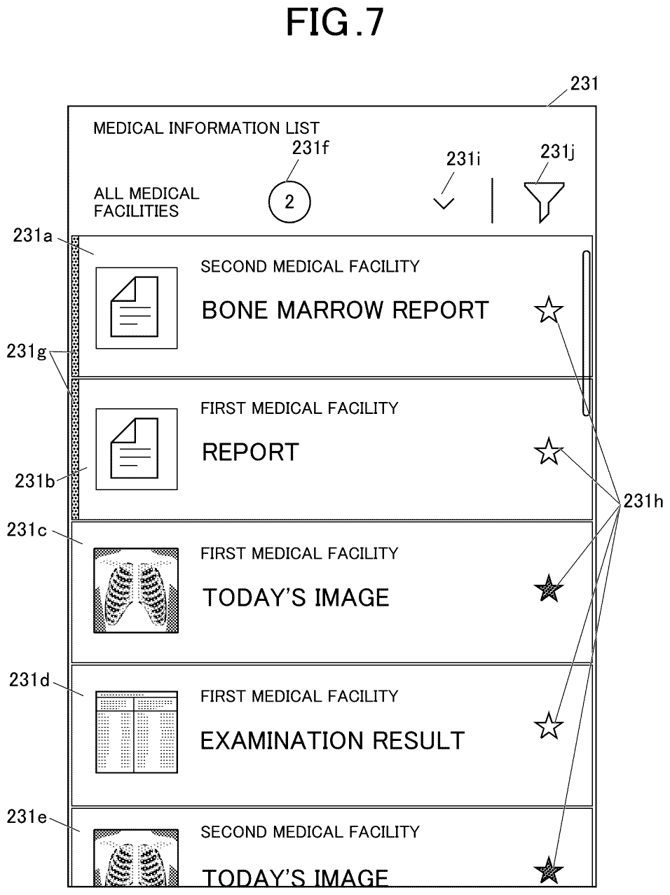

FIG. 7 shows an example of a medical information list screen 231 displayed by the display part 23. The controller 21 causes a list of pieces of medical information at respective medical facilities on the patient using the user terminal 20 stored in the storage section 14 of the information management server 10 to be displayed on the medical information list screen 231. The list of pieces of medical information displayed on the medical information list screen 231 may include medical information at a medical facility (for example, a third medical facility) other than the first medical facility and the second medical facility.

In the example illustrated in FIG. 7, the list of pieces of medical information includes diagnosis result information 231a, diagnosis result information 231b, medical image 231c, examination result information 231d, and medical image 231e.

The diagnosis result information 231a is diagnosis result information (for example, a bone marrow report) as the second medical information obtained by the information management server 10 from the second information terminal 40B of the second medical facility.

The diagnosis result information 231b is diagnosis result information (for example, a report) as the first medical information obtained by the information management server 10 from the first information terminal 40A of the first medical facility.

The medical image 231c is a first medical image as the first medical information obtained by the information management server 10 from the first medical image management apparatus 30A of the first medical facility.

The examination result information 231d is examination result information as the first medical information obtained by the information management server 10 from the first information terminal 40A of the first medical facility.

The medical image 231e is a second medical image as the second medical information obtained by the information management server 10 from the second medical image management apparatus 30B of the second medical facility.

The controller 21 causes an unread count 231f to be displayed on the medical information list screen 231.

The unread count 231f indicates the number of pieces of medical information that are displayed on the medical information list screen 231 and that have not been unread by the patient.

The controller 21 causes, on the medical information list screen 231, the medical information that has not been read by the patient to be displayed in a display manner different from the medical information that has been read by the patient. In the example shown in FIG. 7, the controller 21 adds unread marks 231g to the diagnosis result information 231a and the diagnosis result information 231b that have not been read by the patient.

The controller 21 receives a user's favorite setting of medical information on the medical information list screen 231. The user can set, as a favorite, medical information corresponding to each favorite setting button 231h included in the medical information list screen 231 by pressing the favorite setting button 231h.

The controller 21 receives a press operation on a selection button 231i included in the medical information list screen 231 by the user via the operation part 22.

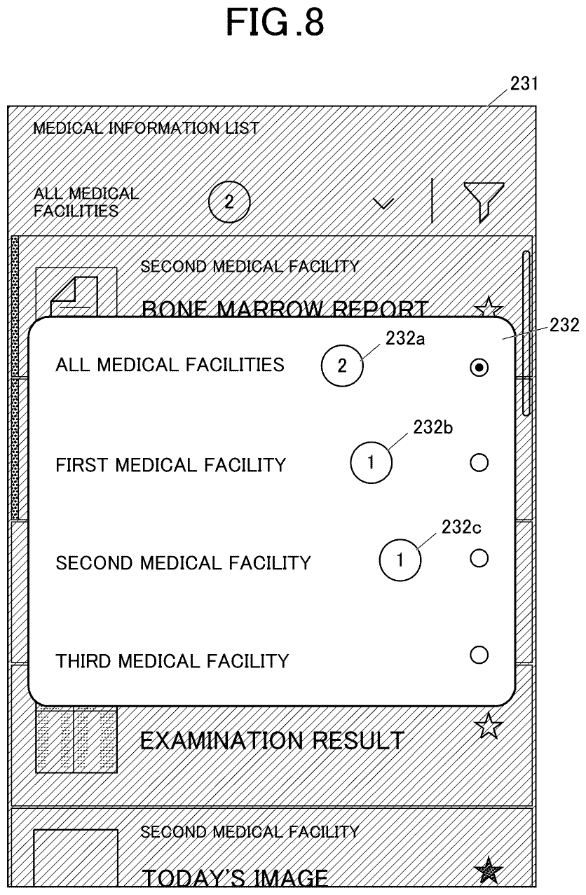

Upon receiving the press operation on the selection button 231i by the user, the controller 21 causes a medical facility selection screen 232 illustrated in FIG. 8 to be displayed on the medical information list screen 231.

The controller 21 receives, on the medical facility selection screen 232, a selection operation to select a medical facility (ies) by the user via the operation part 22. The user selects all the medical facilities or any one of the medical facilities on the medical facility selection screen 232.

When the user selects all the medical facilities, the controller 21 causes a list of pieces of medical information at all the medical facilities on the patient using the user terminal 20 stored in the storage section 14 to be displayed on the medical information list screen 231.

When the user selects one of the medical facilities, the controller 21 displays a list of pieces of medical information at the selected medical facility on the patient using the user terminal 20 stored in the storage section 14 to be displayed on the medical information list screen 231.

In the example illustrated in FIG. 8, an unread count 232a included in the medical facility selection screen 232 indicates the number of pieces of medical information unread by the patient using the user terminal 20 among pieces of medical information at all the medical facilities stored in the storage section 14.

An unread count 232b included in the medical facility selection screen 232 indicates the number of pieces of medical information unread by the patient using the user terminal 20 among pieces of medical information at the first facility stored in the storage section 14.

An unread count 232c included in the medical facility selection screen 232 indicates the number of pieces of medical information unread by the patient using the user terminal 20 among pieces of medical information at the second facility stored in the storage section 14.

The controller 21 receives a press operation on a narrowing-down button 231j included in the medical information list screen 231 by the user via the operation part 22.

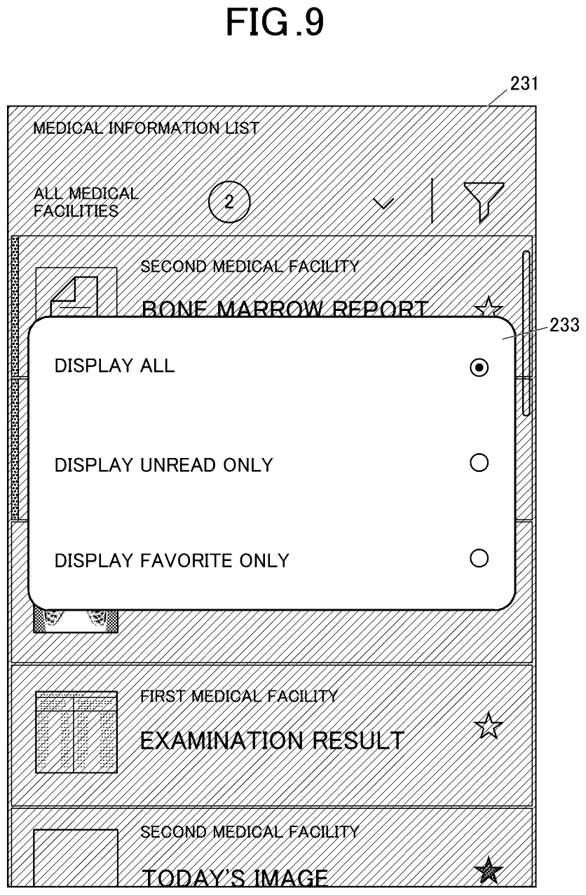

When the controller 21 receives the press operation on the narrowing-down button 231j by the user, the controller 21 causes a narrowing-down screen 233 illustrated in FIG. 9 to be displayed on the medical information list screen 231.

The controller 21 receives, on the narrowing-down screen 233, a selection operation to select a narrowing-down condition for medical information by the user via the operation part 22. The user selects one of “Display All”, “Display Unread Only”, and “Display Favorite Only” on the narrowing-down screen 233.

When the user selects “Display All”, the controller 21 causes a list of all the pieces of medical information at each medical facility on the patient using the user terminal 20 stored in the storage section 14 to be displayed on the medical information list screen 231.

When the user selects “Display Unread Only”, the controller 21 causes a list of unread pieces of medical information among pieces of medical information at each medical facility on the patient using the user terminal 20 stored in the storage section 14 to be displayed on the medical information list screen 231.

When the user selects “Display Favorite Only”, the controller 21 causes a list of favorite-set pieces of medical information among pieces of medical information at each medical facility on the patient using the user terminal 20 stored in the storage section 14 to be displayed on the medical information list screen 231.

That is, the controller 21 causes the display part 23 to display the first medical information and the second medical information satisfying a predetermined condition.

The controller 21 receives, as a setting of the predetermined condition, at least one of a selection of a medical facility (ies), a selection of unread medical information, and a selection of favorite-set medical information. The controller 21 functions as a reception section.

The controller 21 may cause the list of pieces of medical information to be displayed on the medical information list screen 231 in order of importance on the basis of preset importance for pieces of medical information.

Next, the controller 21 receives a selection operation to select one of pieces of medical information on the medical information list screen 231 by the user via the operation part 22. Upon receiving the selection operation for one piece of the medical information by the user, the controller 21 causes the display part 23 to display a screen (not illustrated) where the piece of the medical information selected by the user is displayed on the basis of the display information received from the information management server 10 (Step S7), and ends the information sharing process.

Next, a flow of sharing the vital information in the information sharing system 100 will be described.

The controller 21 of the user terminal 20 transmits the vital information to the information management server 10.

Upon receiving the vital information transmitted from the user terminal 20, the controller 11 of the information management server 10 stores the vital information in the storage section 14 in association with the patient ID. The patient ID is the patient ID indicating the patient using the user terminal 20.

Next, the controller 11 transmits the vital information to the first information terminal 40A and the second information terminal 40B.

Upon receiving the vital information transmitted from the information management server 10, the controllers 41 of the first information terminal 40A and the second information terminal 40B store the vital information in their storage sections 46.

### [Others]

In the above embodiment, the information sharing system 100 may not include the first information terminal 40A and the second information terminal 40B.

In this case, in the information sharing process, Step S2 is omitted.

In the information sharing process in this case, in Step S3, the controller 11 obtains the first medical image which is the first medical information transmitted from the first medical image management apparatus 30A and obtains the second medical image which is the second medical information transmitted from the second medical image management apparatus 30B.

In the information sharing process in this case, in Step S6, the controller 21 causes a list of medical images at each medical facility of the patient using the user terminal 20 stored in the storage section 14 to be displayed on the medical information list screen 231.

In the above embodiment, the information sharing system 100 may not include the first information terminal 40A, and the first medical image management apparatus 30A may have the first medical information other than the first medical image.

In this case, in the information sharing process, in Step S2, the controller 31 of the first medical image management apparatus 30A transmits the first medical information other than the first medical image of the patient using the user terminal 20 to the information management server 10.

In this case, in the flow of sharing the vital information in the information sharing system 100, the controller 11 transmits the vital information to the first medical image management apparatus 30A and the second information terminal 40B.

Upon receiving the vital information transmitted from the information management server 10, the controller 31 of the first medical image management apparatus 30A stores the vital information in the storage section 36. Upon receiving the vital information transmitted from the information management server 10, the controller 41 of the second information terminal 40B stores the vital information in the storage section 46.

### [Effects]

As described above, the information management server 10 of the present embodiment includes the obtainment section (controller 11) that obtains the first medical information in the DICOM format of the first medical facility and the second medical information in the DICOM format of the second medical facility.

The information management server 10 of the present embodiment includes the generation section (controller 11) that generates display information for the display part 23 of the user terminal 20 to display the first medical information and the second medical information obtained by the obtainment section.

The information management server 10 of the present embodiment includes the output section (controller 11) that outputs the display information generated by the generation section.

Thus, the patient can view the first medical information in the DICOM format of the first medical facility and the second medical information in the DICOM format of the second medical facility on the display part 23 of the user terminal 20. Therefore, the patient does not need to take a picture of his/her medical information displayed on a terminal of a doctor with a mobile phone or the like. Since the medical image(s) that the patient can view is medical information in the DICOM format, the image quality is guaranteed. As a result, the patient can appropriately view (pieces of) the medical information from a plurality of medical facilities.

In the information management server 10 of the present embodiment, the obtainment section (controller 11) obtains the first medical information from the first medical image management apparatus 30A of the first medical facility, and obtains the second medical information from the second medical image management apparatus 30B of the second medical facility.

Thus, the patient can view, on the display part 23 of the user terminal 20, the first medical image generated at the first medical facility and the second medical image generated at the second medical facility.

The information sharing system 100 of the present embodiment includes the information management server 10 and the user terminal 20.

The user terminal 20 includes the display part 23 and the controller 21 that causes the display part 23 to display the first medical information and the second medical information satisfying a predetermined condition.

Thus, the patient can view the first medical information and the second medical information satisfying a predetermined condition on the display part 23 of the user terminal 20.

In the information sharing system 100 of the present embodiment, the user terminal 20 includes the reception section (controller 21) that receives, as a setting of the predetermined condition, at least one of a selection of a medical facility (ies), a selection of unread medical information, and a selection of favorite-set medical information.

Thus, the patient can view the first medical information and the second medical information satisfying a desired condition on the display part 23 of the user terminal 20.

Although one or more embodiments of the present invention have been described above, the present invention is not limited to the embodiments, and it is needless to say that various modifications can be made without departing from the scope of the present invention.

For example, in the information sharing system 100 of the above embodiment, one medical image management apparatus 30 is used in one medical facility, but this is not a limitation. A plurality of medical image management apparatuses 30 may be used in one medical facility.

In the information sharing system 100 of the above embodiment, one information terminal 40 is used in one medical facility, but this is not a limitation. A plurality of information terminals 40 may be used in one medical facility.

Besides, the detailed configuration and detailed operation of each part constituting the information management system can also be appropriately modified without departing from the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-183921, filed on Oct. 26, 2023, including description, claims, drawings and abstract is incorporated herein by reference.

What is claimed is:

1. An information management server comprising a hardware processor that
obtains first medical information in a DICOM format of a first medical facility and second medical information in the DICOM format of a second medical facility,
generates display information for a display of a user terminal to display the obtained first medical information and second medical information, and
outputs the generated display information.

2. The information management server according to claim 1, wherein the hardware processor obtains the first medical information from a first medical image management apparatus of the first medical facility and obtains the second medical information from a second medical image management apparatus of the second medical facility.

3. An information sharing system comprising:
the information management server according to claim 1; and
the user terminal including:
the display; and
a hardware processor that causes the display to display the first medical information and the second medical information satisfying a predetermined condition.

4. The information sharing system according to claim 3, wherein the hardware processor of the user terminal receives, as a setting of the predetermined condition, at least one of a selection of a medical facility, a selection of unread medical information, and a selection of favorite-set medical information.

5. A non-transitory computer-readable storage medium storing a program causing a computer of an information management server to perform:
obtaining first medical information in a DICOM format of a first medical facility and second medical information in the DICOM format of a second medical facility;
generating display information for a display of a user terminal to display the obtained first medical information and second medical information; and
outputting the generated display information.
